(19) United States
(12) Patent Application Publication
(10) Pub. No.: US 2003/0077257 A1
(43) Pub. Date: Apr. 24, 2003

(54) Method for preventing and curing diseases of an immune system and a remedy for carrying out said method

(76) Inventor: Vasily Alexandrovich Britov, Vladivostok (RU)

Correspondence Address: Timothy E Newholm, Boyle Fredrickson Newholm Stein & Gratz, Suite 1030, 250 E Wisconsin Avenue, Milwaukee, WI, 53202, US

(21) Appl. No.: 10/204,483

(22) PCT Filed: Jun. 25, 2001
(86) PCT No.: PCT/RU01/00250

(30) Foreign Application Priority Data
Jul. 27, 2000 (RU) 2000120081

(51) Int. Cl. A61K 39/002; A01K 67/033

(52) U.S. Cl. 424/93.7; 424/265.1; 800/8

(57) ABSTRACT

The invention relates to medicine, in particular to methods and remedies for preventing and curing diseases of a human being and an animal, whereby said diseases are associated with a malfunction of the immune system, such as, for example, cancer of the first and second stages and of various localizations or AIDS-infection. The aim of the invention is to increase the efficiency of preventing and curing the immune system of the human being and animal, and to produce active and stable cellular immunity with respect to these diseases. The inventive method for preventing and curing the diseases of the immune system consists in an oral dose of a drug whose a main ingredient contains from 1000 to 6000 alive larvae of the 1895 class of Trichinelia Railliet nematodes, which is specially raised. Said larvae has reduced pathogenic and strong immunogenic properties. The animals infected with the larvae have an easier reconvalescence, and recontamination prevents the disease. The usual therapeutic correction, like for trichinosis, is carried out after the administration of the preparation. The reutilization of the preparation is carried out not before than after 2 months.

### TECHNICAL FIELD

The invention relates to medicine, in particular, to the methods and remedies for preventing and curing diseases of a human being and an animal, whereby the said diseases are associated with a malfunction of the immune system, such as, for example, cancer of the first and second stages of various localizations or AIDS-infection.

### BACKGROUND ART

The methods and remedies for preventing and curing diseases of a human being and an animal are known, that are based on the Trichinella nematode's antigen properties of producing within the host organism the cellular-type immunity to the said diseases.

The way of preventing and curing for immune deficits, such as cancer, for example, is implemented by vaccinations with a sophisticated complex of antigens of the nematodes of the genus Trichinella Raillet, 1895. The vaccine is a specially selected Trichinella line, of the genus Trichinella Raillet, 1895, that is taken per-orally [the Russian Federation Patent Application No. 96123906, published on the 20.03.99].

According to the methods of preventing and curing for diseases of immune system, the vaccination is carried out with larvae Trichina, also taken from a line of Trichinella nematodes specially generated by selection. One dose of this vaccine contains from 3,000 up to 6,000 of larvae. The vaccination can be carried out either once or twice, with an interval of 2.3 days [V. A. Britov, Trichinella as an Immune Stimulator (Thesis of the 9^{th} International Conference on Trichinelliasis, Mexico-City, Mexico, Aug., 19-22, 1996. Page 269.)].

The efficiency of the methods that are known for now is not sufficient for prophylaxis and treatment.

### DISCLOSURE OF THE INVENTION

The aim of the invention is increasing the efficiency of preventing and curing for diseases of immune system of a human being or an animal and creation of active and stable cellular immunity to these diseases.

The said aim is achieved in the following way.

The method of preventing and curing for diseases of immune system suggests per-oral incorporation of the preparation, of which the main ingredient are from 1,000 up to 6,000 larvae Trichina of the genus Trichinella Raillet, 1895 (one dose), that has low pathogenic and high immunogenic properties. After the incorporation of the preparation the ordinary therapeutic correction, like in the case of trichinelliasis, is carried out. And not before than after 2 months, incorporation of another dose of the preparation is carried out for additional induction of the immune-competent cells. For the second incorporation the dose of the preparation can contain more larvae Trichina because the organism already has immunity against Trichinelliasis.

The number of the larva Trichina containing in a dose is found taking into consideration the type of the disease, the patient's age, gender, condition, existence of other diseases and the aim of the immunization (preventing or curing).

The means for preventing and curing for immune system diseases is a preparation whose main part are from 1,000 up to 6,000 larvae Trichina of the genus Trichinella Raillet, 1895 (one dose), that has low pathogenic and high immunogenic properties.

It is better if the larva Trichina are kept in physiological solution.

The action of the preparation is as follows:

After the per-oral incorporation of the preparation, the larva Trichina penetrate into the mucous membrane of the small intestine (intestinum tenue) where they take the form of imago, after that the females produce new larva which are spread throughout the whole organism with the flow of blood. The larva settle inside skeleton muscles throughout the whole body. During their development, the imagos and the larva produce many complex antigens. Under their action, concurrently with formation of the specific mono-immunity to Trichinelliasis, the stronger non-specific immunity develops (due to the cellular immunity) and the organism acquires long-term resistance to other diseases. For example, cancer cellules decompose and the ill parts of organs are replaced by regenerated cellules of these organs or by the conjunctive tissue. The acquired immunity of a human being preserves after the second immunization for up to 8 years.

The preparation is produced of larva of a specially selected Trichinella line, containing in skeleton muscles of trichinous animals. Though there is not any method for direct quantitative characterization of toxic and active properties of trichinellas and their larva, the choice of the line of trichinella that has low pathogenic and high immunogenic properties is carried out experimentally, i.e. by means of incorporation of an experimental dose of Trichinella in immunized animals. For producing the preparation the Trichinella line is chosen that, upon trichinization of laboratory animals, yields in the easiest form of the disease for the animals (rats, for example), and second trichinization does not cause illness.

### THE BEST EXAMPLE FOR CARRYING OUT THE INVENTION

The invention is characterized by the excerption from the following case record (a basal-cellular cancer).

The patient K, born in 1946.

He relates the beginning of his disease with squeezing an acne out from his nose back in 1992. A small wound covered with a peel occurred at the place of the light trauma, it gradually grew. In 1993 he was checked-up by an oncologist, a biopsy analysis were carried out.

The diagnosis was: basalioma of the nose back skin. In 1993 the basalioma was removed by a laser method, after that a relapse began. The tumor size gradually grew, the wound involved more and more area of the nose skin. In 1996 an X-ray therapy was carried out in Khabarovsk, COD 60 TP (epithelization of the X-ray wound). The wound had been closed for a year, then it began to grow and get red. After an occasional strike by a branch of a tree, a wound occurred again that began to grow in size, accompanied by inflammation.

During that period he noticed fatiguability, indisposition, frequent attacks of maxillary sinusitis, angina up to 8 times a year, frequent colds, hemorrhoids, headaches.

The first dose of the preparation was per-orally incorporated in April, 1998. The dose content was: about 2,500 larva of Trichinella in 2 mg. of physiological solution. In addition, the medicine “Detox” (by the “Vision” company) was prescribed and an ointment containing propolis. Within one month after the administration of the preparation the patient's general health improved, the wound on the nose closed.

For the second administration of the preparation, in October 1998, the dose contained about 3,000 of Trichinella larva in 2 mg. of physiological solution,+“Detox”.

The patient came through administration of the both doses of the preparation without any complications. In addition, there were not hemorrhoids attacks any more. During the winter of 1999-2000 his weight increased by 5 kg. The only attack of maxillary sinusitis was in November, 1999, after he overcooled significantly, of which he recovered in 9 days.

The former wound on the nose does not disturb him and until now did not open any more.

The patient has intent, and wants, to incorporate the preparation for the third time, because the positive results are significant, what is obvious from improvement of his general health, increase of immunity, with a prominent anti-cancer action.

### INDUSTRIAL APPLICABILITY

Using the way and the means delivered here, more than 1,000 patients have been successively recovered from different oncologycal and similar diseases, the efficiency of the treatment came to more than 80% and the efficiency of preventing came to more than 95%.

I claim:

1. The way of preventing and curing for diseases of immune system by means of per-oral incorporation of the preparation, of which the main component are nematodes of the genus Trichinella Raillet, 1895, that differs in that incorporation is carried out of the doses of the preparation that contents from 1,000 up to 6,000 alive larva of nematodes of the specially selected line, that has low pathogenic and high immunogenic properties, by which trichinization of immunized animals yields in the easiest form of going through the disease and the second trichinization does not lead to the disease, that is followed by the ordinary therapeutic correction, like for Trichinelliasis, and, not before than after 2 months, the second incorporation of another dose of the preparation is carried out.

2. Method of claim, that differs in that the number of the larvae that the dose contains, is specially chosen, depending on the disease, the patient's age, gender and condition, existence of other diseases and aims of the immunization.

3. Method of claim 1, that differs in that the dose of the preparation intended for the second incorporation contains more larvae Trichinella.

4. The means for preventing and curing for immune system diseases, in the form of the preparation, of which the main ingredient are alive larva of the nematode Trichinella, that differs in that one dose of the preparation contains from 1,000 up to 6,000 of larvae of the nematode of the specially selected line of nematodes of the genus Trichinella Raillet, 1895, that has low pathogenic and high immunogenic properties, by which trichinization of immunized animals yields in the easiest form of going through the disease, and the second trichinization does not yield in the disease.

5. Means of claim 4, that differs in that the larvae are in the physiological solution.
